(19) United States
(12) Patent Application Publication
(10) Pub. No.: US 2009/0170151 A1
(43) Pub. Date: Jul. 2, 2009

(54) FLOW-THROUGH CELL AND METHOD OF USE

(76) Inventor: Gary Owen Shaw, Kinghorn (GB)

Correspondence Address: NIXON & VANDERHYE, PC, 901 NORTH GLEBE ROAD, 11TH FLOOR, ARLINGTON, VA, 22203, US

(21) Appl. No.: 12/374,129

(22) PCT Filed: Jul. 19, 2007
(86) PCT No.: PCT/GB2007/002749

§ 371 Date: Jan. 16, 2009

(30) Foreign Application Priority Data
Jul. 19, 2006 (GB) 0614297.0

(51) Int. Cl. C12Q 1/04; C12M 1/34

(52) U.S. Cl. 435/34; 435/288.7

(57) ABSTRACT

There is disclosed a flow-through cell comprising a substrate defining a channel, having an inlet and an outlet, at least a portion of the substrate being light-permeable to allow particles within at least a portion of the channel between the inlet and the outlet to be optically detected through the substrate, wherein the flow-through cell comprises liquid-permeable particle retaining means located downstream of the at least a portion of the channel where particles can be optically detected, for allowing the flow of a liquid sample through the channel from the inlet to the outlet while retaining particles from the liquid sample whose dimensions exceed threshold dimensions within the channel, where they can be optically detected. The flow-through cell is particularly applicable to the detection of micro-organisms in drinking water.

### FIELD OF THE INVENTION

The invention relates to a flow-through cell which is useful for the detection of particles in general and micro-organisms in particular.

### BACKGROUND TO THE INVENTION

Issues relevant to the invention will now be discussed with reference to the example application of detecting Cryptosporidium oocysts in drinking water, although the same principles apply to the detection of other particles and other micro-organisms in other media.

It is important for public health to screen drinking water for pathogenic micro-organisms such as the protozoa Cryptosporidium and Giardia Lamblia. Because these micro-organisms can be pathogenic in minute quantities, it is advantageous to provide a highly sensitive test capable of screening large liquid samples.

It is known to detect these protozoa by optical microscopy on dry mounted slides, using fluorescent markers which bind specifically to Cryptosporidium oocysts or Giardia cysts or techniques such as differential interference contrast microscopy. Cryptosporidium oocysts have a diameter of 3 to 7 microns. Giardia cysts are typically 8 to 18 microns long and 5 to 15 microns wide. Manual laboratory microscopy techniques are laborious, particularly when an analyst is looking for a very low concentration of micro-organisms.

An automated technique for scanning a microscope slide and detecting Cryptosporidium oocysts and Giardia Lamblia cysts is described in U.S. Pat. No. 6,005,964 (Reid et al.) However, any micro-organisms in the sample which is to be analysed will be spread out across a large surface area requiring time consuming automatic scanning and increasing the risk that an error will be made.

US Patent Application No. 2004/0201845 (Quist et al.) discloses a method of detecting and identifying micro-organisms in a flow through water sample which uses a laser beam and an arrangement of detectors to detect laser light which is scattered from micro-organisms which pass through a small detect area and identifies micro-organisms from the pattern of light scattering. However, only a small proportion of the micro-organisms which pass through the described apparatus will be identified and there is no mechanism provided to retain the detected micro-organisms, making it difficult to check results.

The present invention aims to provide improved apparatus and methodology for detecting particulate objects in liquid samples, which is particularly applicable to the detection of small concentrations of pathogenic micro-organisms in large volumes of water. Some embodiments of the present invention aim to provide improvements to conventional microscope slides to facilitate the detection of micro-organisms in large volumes of water.

### SUMMARY OF THE INVENTION

According to a first aspect of the present invention there is provided a flow-through cell comprising a substrate defining a channel, having an inlet and an outlet, at least a portion of the substrate being light-permeable to allow particles within at least a portion of the channel between the inlet and the outlet to be optically detected through the substrate, wherein the flow-through cell comprises liquid-permeable particle retaining means located downstream of the at least a portion of the channel where particles can be optically detected, for allowing the flow of a liquid sample through the channel from the inlet to the outlet while retaining particles from the liquid sample whose dimensions exceed threshold dimensions within the channel, where they can be optically detected.

The liquid-permeable particle retaining means functions to retain particles whose dimensions exceed threshold dimensions, but to allow liquid to pass through. The liquid-permeable particle retaining means may comprise a size exclusion filter. Preferably, the liquid-permeable particle retaining means are cell and/or micro-organism retaining means.

Thus, particles such as micro-organisms can be retained within the channel where they can be optically detected by optical detection means. Particles, such as micro-organisms can thereby be concentrated from a large volume sample. This can improve the sensitivity of the technique and/or its efficiency in analysing large volume samples. The presence of liquid-permeable particle retaining means may allow other liquids to be passed through the channel, after the sample, without loss of particles, to enable a variety of analytical procedures. For example, a stain or label, such as an immunofluorescent label, may be passed through the channel, from the inlet to the outlet, optionally followed by a wash step, allowing retained particles, such as micro-organisms, to be stained or labelled.

The presence of liquid-permeable particle retaining means may also enable the flow-through cell to be retained to provide a record of particles identified in a particular sample. This allows a sample to be reanalysed at a later stage.

The particles may be cells (such as mammalian tissue cells). Preferably, the particles are micro-organisms, for example Cryptosporidium oocysts or Giardia cysts.

The substrate may define a plurality of such channels. As a result, a liquid sample can be passed through the or each channel.

Optical detection means (discussed below) can be used to detect particles, such as micro-organisms, within the relatively confined space of the or each channel. Advantageously, the flow-through cell may be suitable for analysis through an optical microscope. The flow-though cell may be configured to be usable as a microscope slide. Accordingly, the substrate and/or the flow-through cell as a whole, may be substantially planar and the substrate preferably has parallel first and second principle surfaces. Preferably, the channels run substantially parallel to the first and second principle surfaces. Preferably, the channels are co-planar. Preferably, the substrate extends continuously between the channels. This arrangement reduces or removes discontinuities which might affect the imaging of the substrate through a microscope. The inlets may be located on one of the principle surfaces. The inlets may be located on an edge of the substrate.

The flow-through cell may be a microscope slide. The flow-through cell may be substantially circular and, preferably, the flow-through cell is a circular microscope slide.

Preferably, light can pass through the substrate from the first surface to the second surface. This facilitates optical analysis through the substrate. The substrate may be entirely light permeable, for example the substrate may be entirely transparent.

Preferably, the substrate defines a plurality of channels having an inlet and an outlet. More than one channel may share the same inlet and/or the same outlet, however, each channel preferably has a separate inlet. Preferably also, each channel has a separate outlet. A or each inlet may comprise an elongate hole which is orthogonal to the channel and/or parallel to the thickness of the substrate.

Preferably, the outlets of a plurality of channels (typically all of the channels) open onto different regions of the same liquid-permeable particle retaining means. The liquid-permeable particle retaining means is preferably removable. This enables retained particles to be separated from the flow-through cell and studied.

Preferably, the inlets of the plurality of channels are spaced apart in a regular pattern. This facilitates automatic dispensation of samples into the inlets. The channels may be spaced apart in a regular pattern.

Preferably, the inlets of the plurality of channels are spaced angularly around a centre of rotation of the substrate. The plurality of channels may be spaced angularly. The inlets of the plurality of channels may be in a rotationally symmetric arrangement around a centre of rotation. The plurality of channels may be in a rotationally symmetric arrangement around a centre of rotation.

The substrate may comprise a central opening and the outlets of the plurality of channels may connect to the central opening. The central opening may be an opening in one face of the substrate only. The central opening may comprise wicking means.

The flow-through cell may be adapted to draw a liquid sample into the flow-through cell. To this effect, the or each channel preferably has at least one capillary dimension. Preferably, the channel has a cross-section of 10 to 100 microns in at least one dimension. More preferably, the channel has a cross-section of 30 to 60 microns in at least one dimension. The channel may be circular. The channel may be rectangular. The channel may taper such that it is narrower towards the outlet.

The flow-through cell preferably comprises wicking means (such as a wick) to draw a liquid sample into the or each channel. Typically, the wicking means are in liquid communication with the outlet of the or each channel. Suitable wicking means (such as a wick) may function both as wicking means and as the liquid-permeable particle retaining means.

However, liquid-permeable particle retaining means may be located upstream (that is to say, further towards the inlet) of the wicking means. For example, wicking means may have a filter membrane or layer applied thereto. The substrate may comprise a central opening to which the outlets of the plurality of channels connect and the central opening may comprise wicking means and a filter member or layer located between the outlets and the wicking means. Preferably, the wicking means is operable to wick liquid from the outlets of a plurality of channels.

The wicking means (e.g. a wick) may be removeable. Where there are separate liquid-permeable particle retaining means and wicking means, the liquid-permeable particle retaining means and wicking means are preferably joined to each other and removeable together.

Preferably, the removeable wicking means is in the form of a removeable plug, optionally with the liquid-permeable particle retaining means formed as a layer on an external surface thereof. The removeable plug may have ribbed sides to grip an opening in the substrate.

Where the or each channel has at least one capillary dimension and the flow-through cell comprises wicking means, a liquid sample will be drawn into the channel initially by capillary action and then continue to be drawn through by wicking.

The or each channel is preferably enclosed. The or each channel may be enclosed along some of their length, but be open at the outlet end, with wicking means in contact with at least some of the open portion. Where a plurality of outlets are in liquid communication with the same wicking means, this can reduce cross-contamination between channels.

The substrate may comprise first and second substrate portions which together define the or each channel. Preferably, the first and second substrate portions comprise planar surfaces in contact with each other. One of the substrate portions may comprise one or more elongate indentations which, in combination with the other substrate portion, defines one or more enclosed channels. One of the substrate portions may include one or more grooves on a surface thereof which, in combination with the other substrate portion, define the channel or channels. The grooves may have been formed by etching of the substrate. The same substrate portion, or preferably the other substrate portion, may have one or more holes therethrough which function as the inlet or inlets. Preferably, each of the first and second substrate portions are continuous. By providing continuous substrate portions, optical discontinuities which would affect optical analysis are minimised.

The substrate may comprise first and second substrate portions and a third substrate portion in the form of a layer located between the first and second substrate portions, wherein the first, second and third substrate portions together define at least a portion (preferably the whole length of) the or each channel. Preferably the first and second substrate portions have substantially flat surfaces in contact with the third substrate portion. Preferably, the third substrate portion is in the form of a layer of material with one or more gaps which form the or each channel. Preferably, the or each channel is defined by the first and second substrates and walls on either side of the gaps in the third substrate. The material which constitutes the third substrate portion may extend to within the perimeter of a central aperture of the first or second substrate portion.

Typically, the third substrate portion will be applied to one of the first or the second substrate portion and the other of the first or the second substrate portion will be brought into contact with the third substrate portion and bonded to the third substrate portion. The third substrate portion may be applied as a solid layer and then etched or otherwise cut to form the one or more gaps. The third substrate portion may be formed with the one or more gaps. The third substrate portion may be deposited by applying a material to the first or second substrate using an automatically controlled nozzle or print head.

Preferably, the third substrate portion comprises an adhesive material which adheres the first substrate portion to the second substrate portion. The third substrate portion may consist of an adhesive material shaped to define the or each channel in combination with the first and second substrate portions.

The flow-through cell may comprise a locating notch or segment to enable the flow-through cell to be located in a defined orientation on a support (e.g. a turntable). The flow-through cell may comprise a drive notch or lug for cooperating with a corresponding formation on a support (e.g. a turntable) enabling the flow-through cell to be rotated.

According to a second aspect of the present invention, there is provided detection apparatus which comprises a substrate retaining member for retaining a substrate comprising a plurality of channels within at least a portion of which particles are optically detectable, an optical detector having a magnifying lens configured to optically detect particles within a portion of a channel of a retained substrate where particles can be optically detected and either or both an actuator which is operable to move (e.g. rotate) a retained substrate and an actuator which is operable to move the magnifying lens, to align successive channels in turn with the magnifying lens so that particles can be optically detected within successive channels of a said substrate in turn. An actuator may be operable to move (e.g. rotate) the substrate retaining member to thereby move (e.g. rotate) the substrate. An actuator may be operable to move the magnifying lens relative to a retained substrate.

The invention also extends in a third aspect to a system comprising detection apparatus according to the second aspect of the present invention and a flow-through cell according to the first aspect of the present invention.

The detection apparatus may be adapted to detect particles, such as cells and/or micro-organisms, which have been modified, for example, stained or labelled. The detection apparatus may be adapted to detect particles which are fluorescent or which have been stained or labelled with a fluorescent material. For example, the detection apparatus may comprise a source of light for exciting fluorescence within the at least a portion of the channels where particles can be optically detected. Filter means (such as a high pass filter or band-pass filter, such as a Texas Red filter) may be provided for controlling the frequency range of excitation light. The detection apparatus may comprise filter means (such as a low-pass filter or band-pass filter) for selectively measuring light below a particular frequency or within a frequency range. Such light may be light emitted by the fluorescent micro-organisms or fluorescent material associated with the micro-organisms.

The optical detector may be a camera which takes a two-dimensional image of light emitted within a field of view and magnified by the magnifying lens. The field of view may encompass part of only one channel at a time. The field of view may extend across the entire width of a channel. Preferably, the field of view extends across the entire width of a single channel at one time. The optical detector may be a spectral camera which is operable to record spectral signatures in a range of frequency bands.

The detection apparatus may be adapted to detect moving particles. The detection apparatus may be adapted to detect stationary particles. The detection apparatus may be adapted to identify particles by an identification process which takes into account the shape of detected objects.

The detection apparatus may comprise sample filtration means (such as a filter) for filtering a liquid sample before it is introduced to a channel through the inlet of the channel. The sample filtration means may filter out particles above a particular size. This may reduce false positives and may prevent the channel from becoming clogged. The sample filtration means may filter out particles below a particular size. Where the particles are micro-organisms, the sample filtration means will generally filter out particles with a size above and below the typical size range of the micro-organisms which are to be detected. For example, where the particular are micro-organisms, such as Cryptosporidium oocysts, the sample filtration means may filter out particles with a dimension of less than 3 microns or a dimension of greater than 10 microns.

The substrate preferably comprises a plurality of channels having inlets and the detection apparatus preferably comprises means to introduce successive samples to different channels through their inlets. For example, the detection apparatus may comprise automatic means (such as a substrate holder and motor) for moving the flow-through cell. Where the inlets to the channels are in a rotationally symmetric arrangement around a centre of rotation, the means to introduce successive samples to different channels may comprise means to rotate the flow-through cell around the centre of rotation.

The optical detection means is preferably adapted to detect all particles passing through a cross-section of each channel, allowing all particles, such as cells or micro-organisms, within the liquid sample to potentially be detected.

According to a fourth aspect of the present invention there is provided a flow-through cell comprising a substrate defining a plurality of channels, each of which has an inlet and an outlet, at least a portion of the substrate being light-permeable to allow particles within at least a portion of each channel between the inlet and the outlet of the respective channel to be optically detected through the substrate, wherein wicking means (such as a wick) extends between the outlet of a plurality of channels (preferably the outlets of each channel within the substrate) such that the wicking means is operable to draw a liquid sample into the inlet of each of the plurality of channels.

Typically, the wicking means will not be used to draw a liquid sample into more than one channel at a time. However, by providing wicking means which are operable to draw a liquid sample into the inlet of each of the plurality of channels, a single arrangement may be provided to collect liquid which has passed through more than one channel. Each channel may comprise liquid-permeable particle retaining means located downstream of the at least a portion of the respective channel where particles can be optically detected. Thus, after use of the flow-through cell to retain particles, a liquid applied to the wicking means will flow backwards through each of the plurality of channels to detach retained particles from the liquid-permeable particle retaining means. Further optional features correspond to the features discussed above in relation to the first three aspects.

According to a fifth aspect of the present invention there is provided a flow-through cell comprising a substrate defining a plurality of channels, each of which has an inlet and an outlet, at least a portion of the substrate being light-permeable to allow particles within at least a portion of each channel between the inlet and the outlet of the respective channel to be optically detected through the substrate, wherein the substrate comprises an aperture and the outlet of each of the plurality of channels opens into the aperture.

Thus, liquid can be collected from each of the plurality of channels via the aperture. Typically, the substrate is generally circular. Typically, the aperture is located at the centre of the substrate. Typically, the aperture is circular.

Liquid-permeable particle retaining means may be located within the aperture in contact with each channel. Wicking means may be located within the aperture in liquid communication with each channel.

Further optional features correspond to those discussed in relation to the first four aspects. The aperture typically corresponds to the opening described in relation to the first four aspects.

According to a sixth aspect of the present invention there is provided a method for detecting particles (for example, cells and/or micro-organisms) in a liquid sample, the method comprising the steps of introducing the liquid sample into the or a channel of the substrate of flow-through cell according to the first aspect of the present invention, via the inlet, causing the sample to flow through the channel to the outlet, and detecting particles in the at least a portion of the channel where particles can be detected.

The flow-through cell is preferably adapted to draw a liquid sample into the flow-through cell.

Preferably, the or each channel has at least one capillary dimension and capillary action draws the liquid sample and any particles contained within the sample into the portion of the channel where particles can be detected.

Preferably, the flow-through cell comprises wicking means (such as a wick) to wick a liquid sample through the or each channel and the wicking action draws the liquid sample and any particles contained within the sample into the portion of the channel where particles can be detected. The wicking means are typically in liquid contact with the outlets of the channels.

Preferably, the step of detecting particles in a liquid sample comprises the step of using detection apparatus according to the second aspect of the present invention or the system of the third aspect of the present invention. Thus, capillary action and/or wicking action may drawing the liquid sample and any particles contained within the sample under the magnifying lens of the optical detector.

The method may comprise the step of filtering the liquid sample prior to introducing the liquid sample to the inlet of the or a channel using sample filtration means (described above).

The flow-through cell, detection apparatus, system and method are preferably for the detection of Cryptosporidium oocysts and/or Giardia Lamblia cysts.

The detection of particles may comprise the detection of the presence of particles, the absence of particles, and/or the number of particles present. Specific particles or types of particles, such as specific micro-organisms or types of micro-organisms, may be detected.

The method may include the step of taking samples periodically from a liquid supply and introducing them into different (preferably successive) channels of a flow-through cell. The method may include the step of taking samples from different locations and introducing them into different channels of a flow-through cell.

The method may further comprise the step of retaining the flow-through cell for a period of time. The method may further comprise the step of analysing particles, such as cells and/or micro-organisms, retained within a retained flow-through cell at a later time. The method may comprise the step of analysing retained particles in a retained flow-through cell at a later time using an optical microscope.

The method may further comprise the step of removing retained particles from a channel, or a plurality of channels, by applying a liquid to the outlet of the channel, or plurality of channels, to cause liquid to flow backwards through the channel, or plurality of channels, from the outlet to the inlet. This enables retained particles to be subsequently removed for analysis. The liquid may flow to the inlet from where it can be removed with a pipette. Alternatively, the liquid may flow out from the inlet. Where wicking means are present, the liquid may be applied to the outlet of a channel, or the outlets of a plurality of channels, by applying a liquid to the wicking means.

### BRIEF DESCRIPTION OF THE DRAWINGS

An example embodiment of the invention will now be illustrated with reference to the following Figures in which:

FIG. 1 is a cross-section through a system comprising detection apparatus and a flow-through cell according to the present invention;

FIG. 2 is a perspective view of a first example flow-through cell according to the present invention;

FIG. 3 is a plan view of a first substrate portion of the first example flow-through cell;

FIG. 4 is a cross-section through the first substrate portion of FIG. 3 along line A-A;

FIG. 5 is a plan view of a second substrate portion;

FIG. 6 is a cross-section through a second example of a flow-through cell; and

FIG. 7 is plan view of part of the second example of a flow-through cell.

### DETAILED DESCRIPTION OF AN EXAMPLE EMBODIMENT

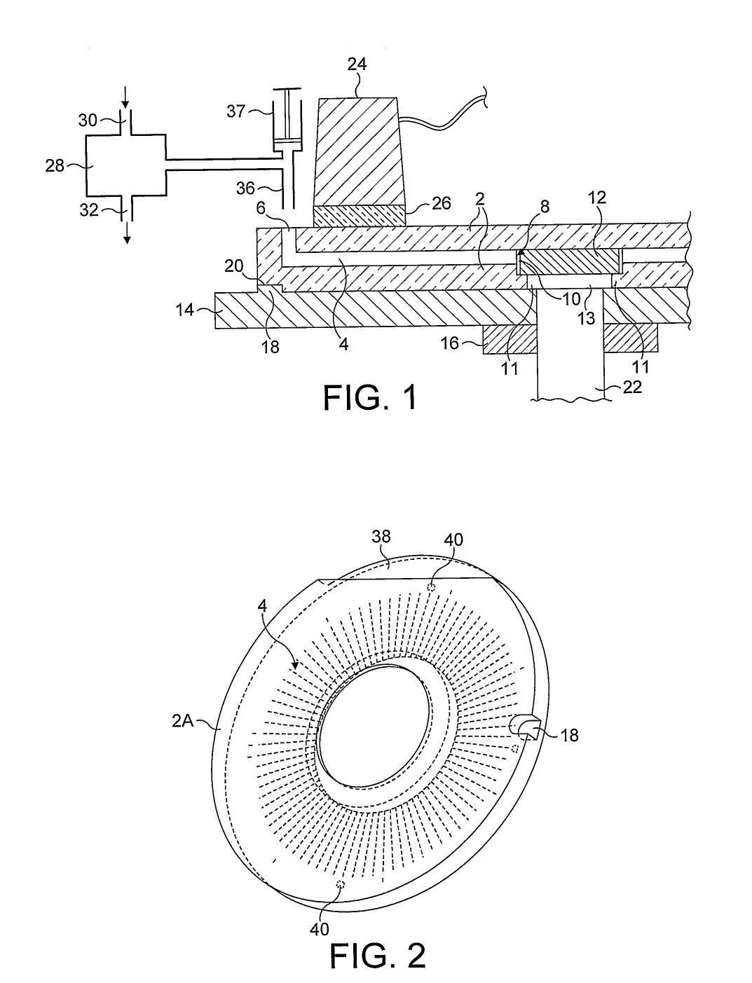

FIG. 1 is a cross-section through a system comprising detection apparatus and a flow-through cell according to the present invention. A flow-through cell 1 comprises a transparent glass substrate 2 which defines a plurality of channels 4, one of which is shown in full. The flow-through cell is made from a high quality optical glass and is substantially planar, allowing it to be used as a microscope slide. Each channel has an inlet 6 and outlet 8. Each channel is around 100 microns wide and 40 microns high. 40 microns is a capillary dimension which causes the substrate to draw a liquid sample introduced through the inlet into the channel.

Each outlet is covered by a size exclusion filter membrane 10 which functions as means to allow the flow of a liquid sample through the channel from the inlet to the outlet while retaining particles (in this case micro-organisms) from the liquid sample whose dimensions exceed threshold dimensions within the channel. A wick 12, such as a borosilicate fibre mat, is located on the other side of the filter membrane, in contact with the filter membrane, so as to draw a liquid sample which has been introduced into the channel through the inlet, and any micro-organisms in the liquid sample, through the channel. The wick is held in place by a ridge 11 around the periphery of a central circular opening 13 in the base of the transparent glass substrate.

The flow-through cell is used in conjunction with detection apparatus. The detection apparatus includes a turntable 14 which supports the flow-through cell in use. The turntable can be rotated under automatic control by a motor 16. The turntable includes a lug 18 which fits into a corresponding notch 20 in the base of the flow-through cell, transmitting drive from the turntable to the flow-through cell. The flow-through cell also includes a segment-shaped cut-out (not shown in FIG. 1) which mates with a cooperating formation on the turntable, to locate the flow-through cell in the correct orientation relative to the turntable. The turntable includes a drain hole 22 through which liquid that has passed through the wick can be drained.

The detection apparatus includes a camera 24 having a magnifying lens 26 which images a region of one channel onto the camera imaging surface (such as a CCD array). The field of view of the camera typically covers the entire width of one channel.

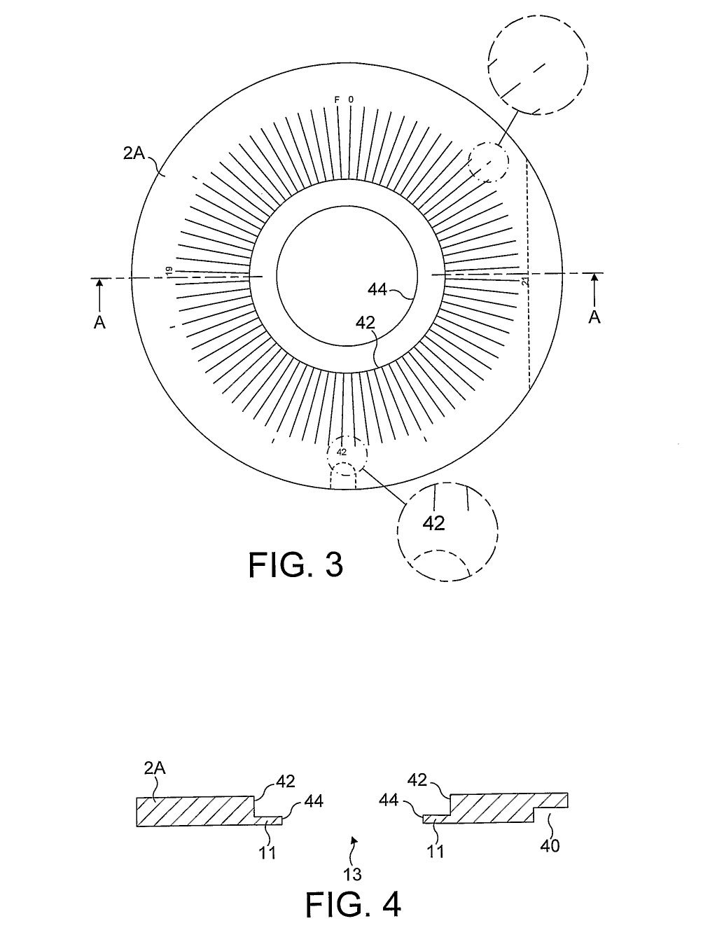

Generally, the detection apparatus will be used with a pre-filter 28 (not to scale) to remove particles which are too large to fit through the channel. The properties of the pre-filter (and dimensions of the channel) are selected so that the pre-filter does not screen out particles of the typical size range of the micro-organism which is to be detected. Typically, the pre-filter will also remove particles below a minimum size by using two separate filters. Where the detection apparatus is used to detect micro-organisms in water, the pre-filter will typically also concentrate the sample and supply a reduced volume liquid sample. Accordingly, the pre-filter will typically comprise an inlet 30 for receiving a liquid sample, a first outlet 32 for removing excess liquid and a second outlet 34 for supplying a reduced volume sample to the flow-through cell. The detection apparatus may include a nozzle 36 for dispensing the liquid sample into the channel inlet and mixing means, such as a syringe 37, for mixing the liquid sample with another liquid, such as a label or stain, before the liquid sample is dispensed into the channel inlet. Typically, the detection apparatus will then rotate the flow-through cell so that a subsequent sample enters the inlet of another channel. FIG. 2 is a perspective view of a first example flow-through cell. In this first example construction, the flow-through cell is manufactured from two substrate portions, each of which is made of transparent glass. FIG. 3 is a plan view of a first substrate portion of the first example flow-through cell.

A plurality of grooves arranged in a rotationally symmetric pattern around the centre of the first substrate portion are etched in a first surface of the first substrate portion. Conveniently, the first substrate portion (and the flow-through cell as a whole) have a diameter of 76.2 mm, which is a conventional diameter for semiconductor wafers, such as silicon wafers, allowing the grooves to be etched using conventional semiconductor wafer patterning and etching techniques.

The first substrate portion includes the notch 18 as well as the segment-shaped cut-out 38 which mates with a cooperating formation on the turntable (not shown), to locate the flow-through cell on the turntable. The first substrate portion conveniently includes markings 40, such as numbers located close to one or more of the grooves, to facilitate identification of the individual grooves. The first substrate portion includes a central bore having a stepped inner circumference. A first inner edge portion 42 located towards the first surface defines a circular space for the wick and filter membrane. A lip 11 including narrower radius second inner edge portion 44 located away from the first surface retains the wick within the flow-through cell. FIG. 4 is a cross-section through the first substrate portion of FIG. 3 along line A-A.

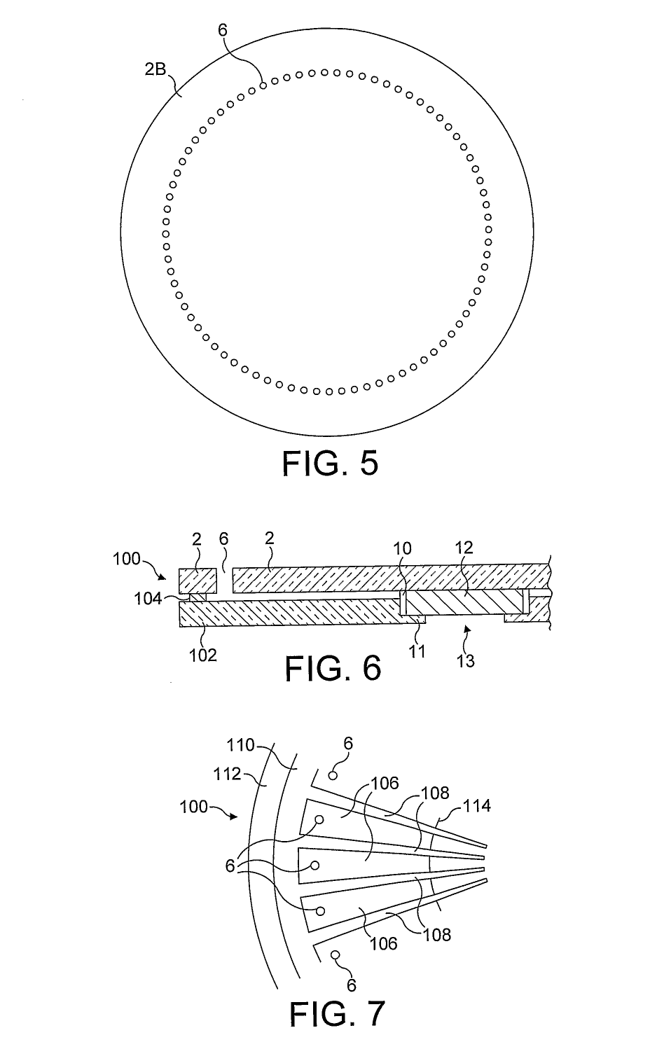

FIG. 5 is a plan view through the second substrate portion 2B. The second substrate portion includes a plurality of holes 6, drilled through the substrate, in a rotationally symmetric pattern around the centre of the second substrate portion. In order to form the flow-through cell, the wick and filter membrane are fitted within the central bore of the first substrate portion and the first and second substrate portions are brought into contact with each other, such that a hole from the second substrate overlies each groove. The substrate portions are then welded to each other by the application of sufficient heat. Thus, the channels are defined by the walls of the grooves and the inlets to the channels are defined by the holes through the second substrate portion.

FIG. 6 is a cross-section through a second example of a flow-through cell 100. As with the first example, the flow-through cell is circular and includes a rotationally symmetric pattern of inlets and channels. The flow-through cell is made from a first substrate portion 102 which corresponds in shape to the first substrate portion of the first example, except that it lacks the rotationally symmetric pattern of grooves, and a second substrate portion 2 which corresponds in shape to the second substrate portion of the first example and includes holes 6 drilled in a rotationally symmetric pattern around the centre of the second substrate portion to function as inlets to channels. A filtration membrane 10 and wick 12 are provided as before. Similarly, the wick is held in place by a ridge 11 around the periphery of a central circular opening 13 in the base of the second substrate portion. However, in the second example, the channels are not defined solely by the first and second substrate portions. A third substrate portion in the form of a layer of adhesive 104 is included between the first and second substrate portions to define the side walls of the channels, with the first and second substrate portions defining the lower and upper walls of the channels respectively.

FIG. 7 is plan view of part of the second example of a flow-through cell including channels 106. The broader upstream part of each channel is located under a hole in the second substrate portion. The third substrate portion is formed from lines of adhesive 108 which extend towards the centre of the flow-through cell from a ring of adhesive 110 in the form of a circle at or near the periphery of the flow-through cell. In the example illustrated in FIG. 7, there is a circular gap 112 around the periphery of the flow-through cell where there is no adhesive. The lines of adhesive which extend towards the centre of the flow-through cell will typically have a constant width, causing the channels to taper in width towards the centre of the flow-through cell. The channels have a vertical capillary dimension as before so that capillary action facilitates drawing liquid into the or each channel.

The lines of adhesive extend beyond the inner circumference 114 (on the side which faces towards the second substrate in use) of the first substrate portion. However, in use, the wick contacts the portions of adhesive which extend beyond the inner circumference. This increases the surface area of wick which is in contact with the channel, which can increase the speed of wicking, and also reduces the risk of cross-contamination between channels. Typically, the filtration membrane will be in contact with the inner circumference 114 of the first substrate portion on the side which faces towards the second substrate in use so that micro-organisms do not penetrate the portion of each channel which extends beyond the inner circumference.

To make the flow-through cell, the adhesive is deposited on the second substrate portion using a nozzle under robotic control. The first substrate portion is then brought into contact with the adhesive layer and thereby bonded to the second substrate portion.

One skilled in the art will recognise that the third substrate portion could be made in many different ways. For example, it may be cut from a piece of material, such as a plastics material, it may be formed as a layer and then etched, it may be printed or deposited by any other means. The first and second substrate portions should be light permeable (and typically transparent) around at least a portion and preferably all of each channel to enable optical detection of micro-organisms within the channel. The third substrate portion may be light permeable.

When the apparatus is used to detect Cryptosporidium oocysts and/or Giardia Lamblia cysts in drinking water, a water sample is first filtered to remove particles which are too large to pass into a channel of the flow-through chamber too small to be the target micro-organism. The sample is also concentrated to reduce the volume of the sample which is introduced into the channel. Ideally, a very large volume of water, e.g. 1,000 litres, will be concentrated to a small sample volume, e.g. 1.5 ml, without loss of micro-organisms.

In a preferred embodiment, micro-organisms are stained with a fluorescent dye such as 4′-6-Diamidino-2-phenylindole (DAPI) prior to being introduced into the flow-through cell. The syringe controlled by a stepper motor takes up a volume of condensed, filtered sample, followed by a further volume of fluorescent dye. After a period of time (e.g. 15 minutes) to allow the dye to stain the micro-organisms, the resulting sample is then introduced into the inlet of a first channel of a flow-through chamber through the inlet. The sample is drawn into the channel by capillary action. Once it contacts the wick, it continues to be drawn through by the wicking action of the wick.

The liquid sample and any micro-organisms within the liquid sample will thereby flow past the magnifying lens, enabling the labelled or stained micro-organisms to be optically detected by the camera. The field of view of the camera extends across the whole width of a single channel. The micro-organisms within the sample will be retained in the channel by the filtration membrane.

After each sample, the detection apparatus causes the turntable to be rotated so that the next sample is introduced into the inlet of the next channel. Thus liquid samples from different locations or different times can be introduced into consecutive channels. For example, a sample may be taken from a drinking water supply every two hours and introduced into consecutive channels. Thus, a flow-through cell with 84 channels could receive samples every two hours for a week.

Importantly, because the micro-organisms are retained within the flow-through cell, the flow-through cell can be stored to keep a record of successive samples. In the event that a water supply is subsequently found to have been contaminated with a micro-organism, the retained flow-through cell can be studied, allowing the change in the level of micro-organisms with time to be studied. Because the flow-through cell is planar and of suitable dimensions for use with an optical microscope, it functions as a microscope slide and so this later analysis can be carried out manually using an optical microscope if desired. The retained micro-organisms may be removed for later analysis by wetting the wick, whereupon liquid flows into the outlet, displacing retained micro-organisms from the filter which flow with the liquid out from the inlet of each channel.

In an alternative embodiment, the filter membrane is formed as a layer around the periphery of a removable wick. The removable wick is generally cylindrical with a peripheral wall formed from a plastics material. The peripheral wall is ridged to enable the removable wick to be detachably retained in the central opening. The removable portion is formed as several generally circular layers. A first layer, which is in contact with the outlets of the channels in use, is hydrophilic and functions as both a wick and a filter. A second layer, in liquid communication with the first layer is made from a fabric wicking material. A third layer, which is larger than the first two layers, is formed from a looser woven fabric wicking material than the second layer. A fourth layer comprises a rigid grid which extends across the base of the removable wick to provide mechanical strength in the event that a vacuum is applied to the removable wick. The removable wick further comprises an RFID tag to facilitate tracking of the removable wick. Accordingly, the removable wick can be stored and used as a record of micro-organisms retained by the filter. In this embodiment, no rim is provided around the periphery of the opening in the base of the transparent glass substrate, so that the wick can be removed.

In a further alternative embodiment, the particle retaining means is removable separately to the wick.

In another embodiment, micro-organisms could be detected without staining or labelling. Micro-organisms could be detected whilst stationery after the liquid sample has passed through the channel, in which case the field of view of the camera will typically be close to the outlet of the channel. In another embodiment, further liquids are passed through the channel prior to detection, for example, the sample may not be stained or labelled prior to being introduced to the channel and a stain or label, such as a fluorescent immunolabel or dye for labelling the micro-organisms, may be subsequently introduced, followed by a wash liquid.

Further modifications and variations may be made within the scope of the invention herein disclosed.

1. A flow-through cell comprising a substrate defining a channel, having an inlet and an outlet, at least a portion of the substrate being light-permeable to allow particles within at least a portion of the channel between the inlet and the outlet to be optically detected through the substrate, wherein the flow-through cell comprises liquid-permeable particle retaining means located downstream of the at least a portion of the channel where particles can be optically detected, for allowing the flow of a liquid sample through the channel from the inlet to the outlet while retaining particles from the liquid sample whose dimensions exceed threshold dimensions within the channel, where they can be optically detected.

2. A flow-through cell as claimed in claim 1, wherein the substrate defines a plurality of said channels.

3. A flow-through cell as claimed in claim 2, wherein the inlets of the channels are arranged in a regular pattern.

4. A flow-through cell as claimed in claim 3, wherein the inlets of the channels are arranged in a rotationally symmetric pattern.

5. A flow-through cell as claimed in claim 1 which is adapted to draw a liquid sample into the flow-through cell.

6. A flow-through cell as claimed in claim 5, wherein the or each channel has a capillary dimension.

7. A flow-through cell as claimed in claim 5, further comprising wicking means to draw a liquid sample into the or each channel.

8. A flow-through cell as claimed in claim 7, wherein the wicking means functions as both the wicking means and the liquid-permeable particle retaining means.

9. A flow-through cell as claimed in claim 1, wherein the substrate defines a plurality of said channels, and the substrate comprises a central opening into which the outlets of the plurality of channels open.

10. A flow-through cell as claimed in claim 9, wherein the walls of the plurality of channels extend into the central opening such that the outlets of the channels are only partially enclosed by the substrate.

11. A flow-through cell as claimed in claim 10, comprising first and second substrate portions, wherein the or each channel was formed by etching a substrate portion.

12. A flow-through cell as claimed in claim 1, comprising first and second substrate portions and a third substrate portion in the form of a layer between the first and second substrate portions, and wherein the first, second and third substrate portions together define at least a portion of the length of the one or more channels.

13. A flow-through cell as claimed in claim 12, wherein the third substrate portion is an adhesive layer.

14. A flow-through cell as claimed in claim 12, wherein the substrate defines a plurality of said channels, and the substrate comprises a central opening into which the outlets of the plurality of channels open, and wherein each channel is enclosed along part of its length

15. A flow-through cell as claimed in claim 1, wherein the particle retaining means is removable.

16. A flow-through cell as claimed in claim 16, wherein the outlets of a plurality of channels open onto different regions of the same removable liquid-permeable particle retaining means.

17. Detection apparatus comprising a substrate retaining member for retaining a substrate comprising a plurality of channels within at least a portion of which particles are optically detectable, an optical detector having a magnifying lens configured to optically detect particles within a portion of a channel of a retained substrate where particles can be optically detected and either or both an actuator which is operable to move a retained substrate and an actuator which is operable to move the magnifying lens, to align successive channels in turn with the magnifying lens so that particles can be optically detected within successive channels of a said substrate in turn.

18. A system comprising detection apparatus as claimed in claim 17, comprising a substrate which is retainable by the substrate retaining member of the detection apparatus.

19. A system as claimed in claim 18, wherein the detection apparatus comprises a source of light for exciting fluorescence within the at least a portion of the channels where particles can be detected.

20. A system as claimed in claim 19, comprising mixing means for mixing a liquid sample with a stain or label before the liquid sample is introduced to a channel.

21. A system as claimed in claim 18, comprising sample filtration means for filtering a liquid sample before it is introduced to a channel through the inlet of the channel.

22. A system as claimed in claim 18 which is adapted to detect micro-organisms and or cells.

23. A flow-through cell comprising a substrate defining a plurality of channels, each of which has an inlet and an outlet, at least a portion of the substrate being light-permeable to allow particles within at least a portion of each channel between the inlet and the outlet of the respective channel to be optically detected through the substrate, wherein wicking means extends between the outlet of a plurality of channels such that the wicking means is operable to draw a liquid sample into the inlet of each of the plurality of channels.

24. A flow-through cell as claimed in claim 23, wherein each channel comprises liquid-permeable particle retaining means located downstream of the at least a portion of the respective channel where particles can be optically detected.

25. A flow-through cell comprising a substrate defining a plurality of channels, each of which has an inlet and an outlet, at least a portion of the substrate being light-permeable to allow particles within at least a portion of each channel between the inlet and the outlet of the respective channel to be optically detected through the substrate, wherein the substrate comprises an aperture and the outlet of each of the plurality of channels opens into the aperture.

26. A flow-through cell according to claim 25, wherein liquid-permeable particle retaining means is located within the aperture in contact with each channel to retain particles within each channel.

27. A flow-through cell according to claim 25, wherein wicking means are located within the aperture in liquid communication with each channel.

28. A method for detecting particles in a liquid sample, the method comprising the steps of introducing the liquid sample into the or a channel of the substrate of flow-through cell according to claim 1, via the inlet, causing the sample to flow through the channel to the outlet, and detecting particles in the at least a portion of the channel where particles can be detected.

29. A method according to claim 28, wherein the flow-through cell is adapted to draw a liquid sample into the flow-through cell.

30. A method according to claim 28, wherein the step of detecting particles in a liquid sample comprises the step of using detection apparatus which comprises an optical detector having a magnifying lens configured to optically detect particles within the at least a portion of the channel where matter can be optically detected

31. A method according to claim 28, wherein the method comprises the step of filtering the liquid sample prior to introducing the liquid sample to the inlet of the or a channel using sample filtration means.

32. A method according to claim 28, comprising the step of mixing the liquid sample with a label or stain prior to introducing the liquid sample into the or a channel.

33. A method according to claim 28 for detecting the presence of particles, the absence of particles, and/or the number of particles present.

34. A method of detecting Cryptosporidium oocysts and/or Giardia Lamblia cysts as claimed in claim 28.

35. A method as claimed in claim 28, comprising the step of taking samples periodically from a liquid supply and introducing them into different channels of the flow-through cell.

36. A method as claimed in claim 28, further comprising the step of removing retained particles from a channel, or a plurality of channels, by applying a liquid to the outlet of the channel, or plurality of channels, to cause liquid to flow backwards through the channel, or plurality of channels, from the outlet to the inlet.

37. A method as claimed in claim 36, wherein the liquid flows to the inlet.

38. A method as claimed in claim 36, wherein the substrate comprises wicking means in liquid communication with the outlet of one or more channels to draw a liquid into the one or more channels, wherein liquid is applied to the outlet or outlets of the one or more channels by applying the liquid to the wicking means.
